(19) United States
(12) Patent Application Publication
(10) Pub. No.: US 2025/0215001 A1
(43) Pub. Date: Jul. 3, 2025

(54) NOVEL METHOD FOR PREPARING RUCAPARIB

(71) Applicant: KOREA UNIVERSITY RESEARCH AND BUSINESS FOUNDATION, Seoul (KR)

(72) Inventors: Cheol-Hong CHEON, Seoul (KR); Jinjae PARK, Seoul (KR)

(73) Assignee: KOREA UNIVERSITY RESEARCH AND BUSINESS FOUNDATION, Seoul (KR)

(21) Appl. No.: 18/846,595

(22) PCT Filed: Jul. 28, 2022
(86) PCT No.: PCT/KR2022/011114

§ 371 Date: Sep. 12, 2024

(30) Foreign Application Priority Data
Aug. 3, 2021 (KR) 10-2021-0101905

(51) Int. Cl. C07D 487/04; C07C 201/12; C07C 231/12; C07C 231/14; C07D 209/14

(52) U.S. Cl. CPC C07D 487/04; C07C 201/12; C07C 231/12; C07C 231/14; C07D 209/14

(57) ABSTRACT

The present disclosure relates to a novel method for preparing rucaparib capable of achieving excellent synthesis yield and reproducibility. In particular, the present disclosure relates to a novel preparation method for synthesizing rucaparib by first synthesizing an indole skeleton to which substituents are introduced to positions 2, 3, 4 and 6, and then performing a heptagonal lactam ring formation reaction between the substituents introduced to positions 3 and 4, and a novel intermediate that may be used in preparation thereof.

### TECHNICAL FIELD

The present disclosure relates to a novel method for preparing rucaparib capable of achieving excellent synthesis yield and reproducibility. In particular, the present disclosure relates to a novel preparation method for synthesizing rucaparib by first synthesizing an indole skeleton to which substituents are introduced to positions 2, 3, 4 and 6, and then performing a heptagonal lactam ring formation reaction between the substituents introduced to positions 3 and 4, and a novel intermediate that may be used in preparation thereof.

### BACKGROUND ART

Rucaparib (trade name: rubraca) is an anticancer drug approved as a treatment for ovarian cancer through poly(ADP-ribose) polymerase (PARP) inhibition in late 2016 by the US FDA. After the approval by the US FDA in 2016, rucaparib was approved for use in Europe as well in 2017, and has been used as a treatment for ovarian cancer through PARP inhibition in the US and five European countries (UK, Germany, France, Italy and Spain) since 2018. In addition to ovarian cancer, rucaparib was also approved for prostate cancer in 2020, and is in extensive preclinical stages for many other types of cancer, including breast cancer.

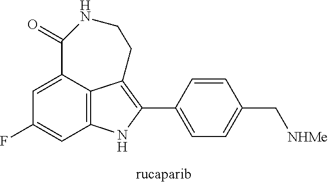 [SMILES: CNCc1ccc(-c2[nH]c3cc(F)cc4c3c2CCNC4=O)cc1]

Currently, rucaparib is mass produced through a synthetic route (Scheme 1) developed by Pfizer Inc. in 2012, however, this synthetic route proceeds through h a linear sequence, and in some steps, synthesis yield is somewhat low and there is a problem with reproducibility. In particular, considering the marketability of rucaparib as a treatment for ovarian cancer and that possibility of rucaparib as a targeted anticancer drug for other types of cancer using a similar anticancer mechanism is investigated, development of a novel synthetic method for a rucaparib compound is necessary.

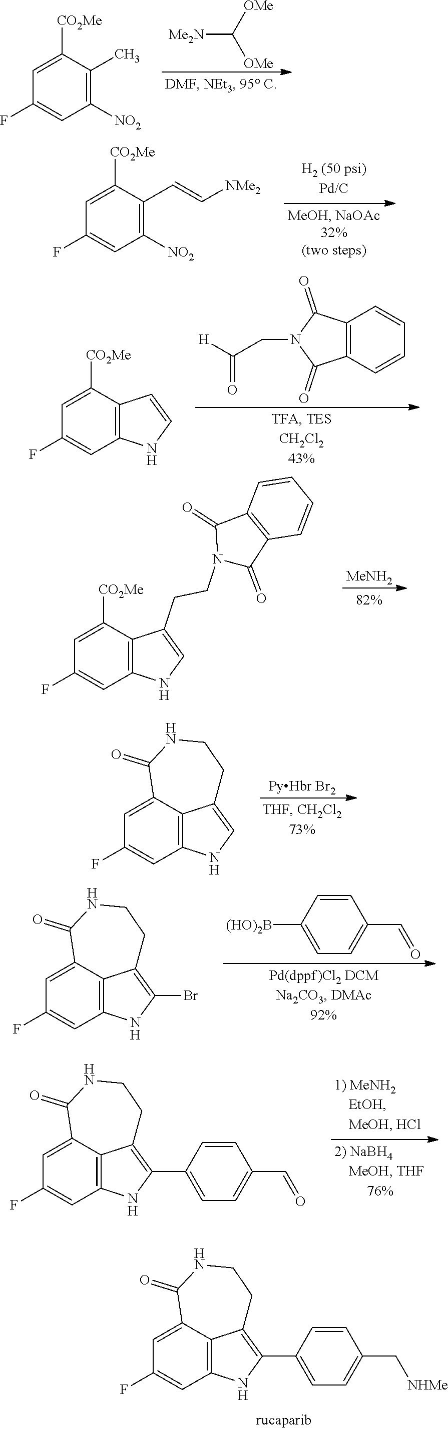

### Scheme 1. Synthetic Route Developed by Pfizer Inc.

Recognizing the need for developing such a novel synthetic method, numerus patents and papers on novel synthetic methods of this anticancer drug have been published after the FDA approval in 2016, however, most of them have focused on the synthesis of indoloazepine compound, a key intermediate in the existing synthetic method, and synthetic methods significantly improving the synthetic route have not been developed.

In view of the above, the inventors of the present disclosure have found that excellent synthesis yield and reproducibility are able to be achieved when synthesizing rucaparib by, unlike the previous synthetic route, first synthesizing an indole skeleton to which substituents are introduced to positions 2, 3, 4 and 6, and then performing a heptagonal lactam ring formation reaction between the substituents introduced to positions 3 and 4, and have completed the present disclosure.

### DISCLOSURE

### Technical Problem

The present disclosure is directed to providing a novel method for preparing rucaparib capable of achieving excellent synthesis yield and reproducibility.

The present disclosure is also directed to providing a novel intermediate that may be used in preparation of rucaparib.

### Technical Solution

In view of the above, an embodiment of the present disclosure provides a method for preparing a compound of Chemical Formula (3), the method including: reacting a compound of the following Chemical Formula (1) and a compound of the following Chemical Formula (2); and converting the result into a compound of the following Chemical Formula (3) in the presence of a catalyst:

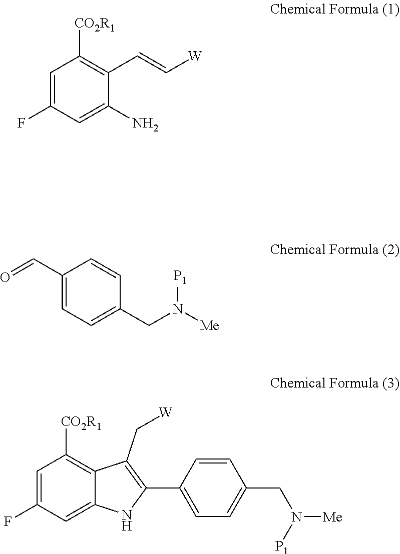

herein,
R₁ is a linear or branched C₁-C₅ alkyl;
W is selected from the group consisting of —COOR₂, —CONH₂, —CONHP₂ and —CONP₂P₃;
R₂ is H or a linear or branched C₁-C₈alkyl; and
P₁, P₂ and P₃ are, as an amine protecting group, each independently selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

The term “C₁-C₅ alkyl” used in the present disclosure means a hydrocarbon having 1 to 5 carbon atoms, and the term “linear or branched” means that the hydrocarbon includes normal, secondary or tertiary carbon atoms. Specifically, proper examples of the “C₁-C₅ alkyl” include methyl, ethyl, 1-propyl (n-propyl), 2-propyl, 1-butyl, 2-methyl-1-propyl, 3-pentyl and the like, but are not limited thereto.

The term “protecting group” used in the present disclosure refers to a moiety of a compound completely shielding or altering properties of a functional group or properties of the compound. Chemical substructures of a protecting group are very diverse. One function of a protecting group is to act as an intermediate in the synthesis of a parent drug substance. Chemical protecting groups and strategies for protection/deprotection are widely known in the related art. Regarding this, literatures [“Protective Groups in Organic Chemistry”, Theodora W. Greene (John Wiley & Sons, Inc., New York, 1991)] and [Protective Groups in Organic Chemistry, Peter G. M. Wuts and Theodora W. Greene, 4th Ed., 2006] are referenced. Protecting groups are often used to shield reactivity of a particular functional group and assist in efficiency of a target chemical reaction. Protection of a functional group of a compound alters other physical properties other than reactivity of the protected functional group, such as polarity, hydrophobicity, hydrophilicity, and other properties that may be measured using common analytical tools. Chemically protected intermediates may be biologically and chemically active or inactive as they are. The term “amine protecting group” refers to a protecting group useful for protecting an amine group (—NH₂).

In a preferred embodiment of the present disclosure, preferred examples of the “amine protecting group” include methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc), however, the amine protecting group is not limited thereto, and protecting groups that may perform a role chemically equivalent to the protecting group are included in the category of the present disclosure.

In an embodiment of the present disclosure, the step of conversion into the compound of Chemical Formula (3) is preferably performed in the presence of a dehydrating agent. Using such a dehydrating agent may facilitate the overall reaction by removing water molecules generated when forming an imine intermediate.

In a preferred embodiment of the present disclosure, preferred examples of the dehydrating agent include at least one compound selected from the group consisting of TiCl₄, MgSO₄ and Na₂SO₄, but are not limited thereto. In addition, the step of conversion into the compound of Chemical Formula (3) may be performed by reacting with molecular sieves, or using an azeotropic distillation method.

In a preferred embodiment of the present disclosure, the catalyst used in the step of conversion into the compound of Chemical Formula (3) is MCN or N-heterocyclic carbene, and herein, M is an alkali metal or NR₄⁺, and R is H or a linear or branched C₁-C₅ alkyl. The catalyst performs a role of facilitating a reaction in which an imine intermediate produced in the middle of the reaction forms an indole skeleton through cyclization.

In a preferred embodiment of the present disclosure, the N-heterocyclic carbene includes a compound selected from the group consisting of imidazolium, triazolium and thiazolium, but is not limited thereto.

A preferred embodiment of the present disclosure provides a preparation method further including, when W is —COOR₂, —CONH₂ or —CONP₂P₃, converting the —COOR₂, —CONH₂ or —CONP₂P₃ into —CONHP₂ after the step of conversion into the compound of Chemical Formula (3):
herein, R₂ is H or a linear or branched C₁-C₅ alkyl; and
P₁, P₂ and P₃ are, as an amine protecting group, each independently selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

In addition, an embodiment of the present disclosure provides a method for preparing rucaparib, a compound of the following Chemical Formula (5), the method including:
(a) reacting a compound of the following Chemical Formula (1) and a compound of the following Chemical Formula (2), and converting the result to a compound of the following Chemical Formula (3) in the presence of a catalyst, however, when W is —COOR₂, —CONH₂ or —CONP₂P₃, converting the —COOR₂, —CONH₂ or —CONP₂P₃ into —CONHP₂ is further included after the step (a);
(b) performing a reaction of reducing the compound of Chemical Formula (3) to convert the result into a compound of the following Chemical Formula (4); and
(c) subjecting the compound of Chemical Formula (4) to a lactam ring formation reaction, and to deprotection before, after or simultaneously with the reaction to obtain a compound of the following Chemical Formula (5):

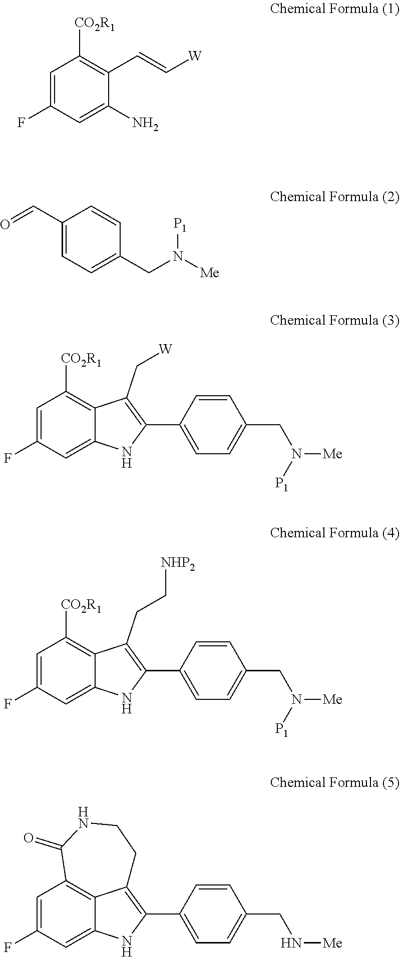

herein,
R₁ is a linear or branched C₁-C₅ alkyl; W is selected from the group consisting of —COOR₂, —CONH₂, —CONHP₂ and —CONP₂P₃;
R₂ is H or a linear or branched C₁-C₅ alkyl; and
P₁, P₂ and P₃ are, as an amine protecting group, each independently selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

In an embodiment of the present disclosure, the reduction reaction of the step (b) is preferably performed in the presence of a metal catalyst selected from the group consisting of Ni, Zn, Fe and Co, and a silane compound, or performed in the presence of a metal hydride selected from the group consisting of DIBAL-H, L-selectride, NaBH₄ and borane.

In a preferred embodiment of the present disclosure, examples of the silane compound used in the present disclosure include PhSiH₃, Ph₂SiH₂, Ph₃SiH, (EtO)₃SiH, Et₃SiH, Me₂SiHSiHMe₂, PMHS (polymethylhydrosiloxane), TMDS (1,1,3,3-tetramethyldisiloxane) and the like, but are not limited thereto.

In a preferred embodiment of the present disclosure, the step (c) performs a lactam ring formation reaction through the amine group produced after deprotecting the compound of Chemical Formula (4) to prepare the compound of Chemical Formula (5).

In a preferred embodiment of the present disclosure, the step (c) is performed in the presence of a base when performing a lactam reaction ring formation before deprotecting the compound of Chemical Formula (4), and deprotects the compound of Chemical Formula (4) after or simultaneously with the reaction to prepare the compound of Chemical Formula (5). Examples of the base that may be used in an embodiment of the present disclosure include strong bases such as M′HMDS (M′=Li, Na, K), LDA and MOtBu (M=Na, K), but are not limited thereto, and all bases capable of performing a dehydrogenation reaction from the compound of Chemical Formula (4) are included in the category of the present disclosure.

An embodiment of the present disclosure provides a method for preparing a compound of Chemical Formula (1), the method including:
(a′) converting a compound of the following Chemical Formula (6) to a compound of the following Chemical Formula (7);
(b′) reacting the compound of Chemical Formula (7) with a compound of the following Chemical Formula (8) to prepare a compound of the following Chemical Formula (9); and
(c′) performing a reaction of reducing the compound of Chemical Formula (9):

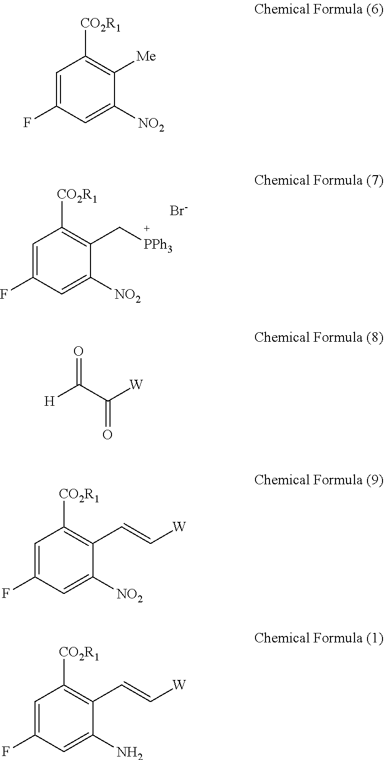

herein,
R₁ is a linear or branched C₁-C₈alkyl;
W is selected from the group consisting of —COOR₂, —CONH₂, —CONHP₂ and —CONP₂P₃;
R₂ is H or a linear or branched C₁-C₅ alkyl; and
P₁, P₂ and P₃ are, as an amine protecting group, each independently selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2, 2,2-trichloroethoxycarbonyl (Troc), 2-trimethylsilylethoxycarbonyl (Teoc) and aryloxycarbonyl (Alloc).

In addition, an embodiment of the present disclosure provides a compound of the following Chemical Formula (1) or a compound of the following Chemical Formula (3) as a novel intermediate that may be used in preparation of rucaparib of the present disclosure:

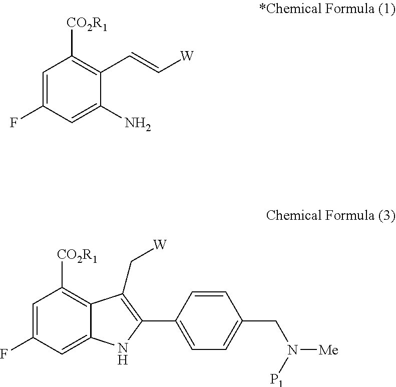

herein,
R₁ is a linear or branched C₁-C₈alkyl;
W is selected from the group consisting of —COOR₂, —CONH₂, —CONHP₂ and —CONP₂P₃;
R₂ is H or a linear or branched C₁-C₅ alkyl; and
P₁, P₂ and P₃ are, as an amine protecting group, each independently selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2, 2,2-trichloroethoxycarbonyl (Troc), 2-trimethylsilylethoxycarbonyl (Teoc) and aryloxycarbonyl (Alloc).

Any suitable solvent may be used in the method of the present disclosure. Representative solvents include pentane, pentanes, hexane, hexanes, heptane, heptanes, petroleum ether, cyclopentane, cyclohexane, benzene, toluene, xylene, dichloromethane, trifluoromethylbenzene, halobenzene such as chlorobenzene, fluorobenzene, dichlorobenzene and difluorobenzene, methylene chloride, chloroform, acetone, acetonitrile, ethyl acetate, diethyl ether, tetrahydrofuran (THF), 2-methyltetrahydrofuran, dibutyl ether, diisopropyl ether, methyl dimethoxyethane, dioxane (1,4 dioxane), N-methyl pyrrolidinone (NMP), DMF, alcohol such as methanol, ethanol, propanol and butanol, or mixtures thereof, but are not limited thereto.

The reaction mixture of each step of the present disclosure may be under any suitable pressure. For example, the reaction mixture may be under atmospheric pressure. The reaction mixture may also be exposed to any suitable environment such as atmospheric gas, or inert gas such as nitrogen or argon.

The reaction of each step of the present disclosure may be performed at any suitable temperature. For example, the temperature of the mixture during the reaction may be from −78° C. to 150° C., −50° C. to 100° C., −25° C. to 100° C., 0° C. to 100° C., room temperature to 100° C., 50° C. to 100° C. or 50° C. to 150° C.

### Advantageous Effects

According to the present disclosure, it is possible to obtain a novel preparation method for synthesizing rucaparib by first synthesizing an indole skeleton to which substituents are introduced to positions 2, 3, 4 and 6, and then performing a heptagonal lactam ring formation reaction between the substituents introduced to positions 3 and 4, and a novel intermediate that may be used in preparation thereof.

### BRIEF DESCRIPTION OF DRAWINGS

FIG. 1 is an NMR spectrum of (E)-N-(4-methoxybenzyl)-2-amino-4-fluoro-6-methoxycarbonylcinnamide (Compound 3).

FIG. 2 is an NMR spectrum of (E)-ethyl 2-amino-4-fluoro-6-methoxycarbonylcinnamate (Compound 9).

FIG. 3 is an NMR spectrum of N-(4-methoxybenzyl)-2-(4-(N-tert-butoxycarbonyl-N-methylaminomethyl)phenyl)-6-fluoro-4-methoxycarbonyl-indole-3-acetamide (Compound 2).

FIG. 4 is an NMR spectrum of ethyl 2-(4-(N-tert-butoxycarbonyl-N-methylaminomethyl)phenyl)-6-fluoro-4-methoxycarbonyl-indole-3-acetate (Compound 10).

FIG. 5 is an NMR spectrum of rucaparib (Compound 1).

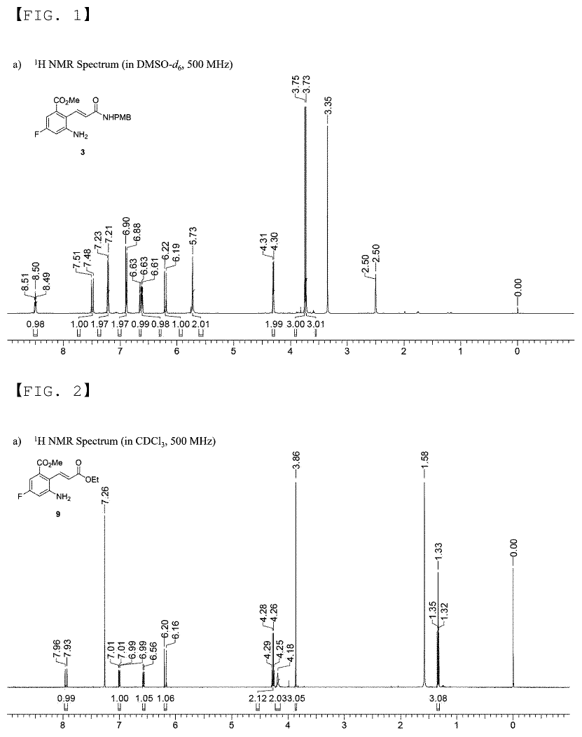

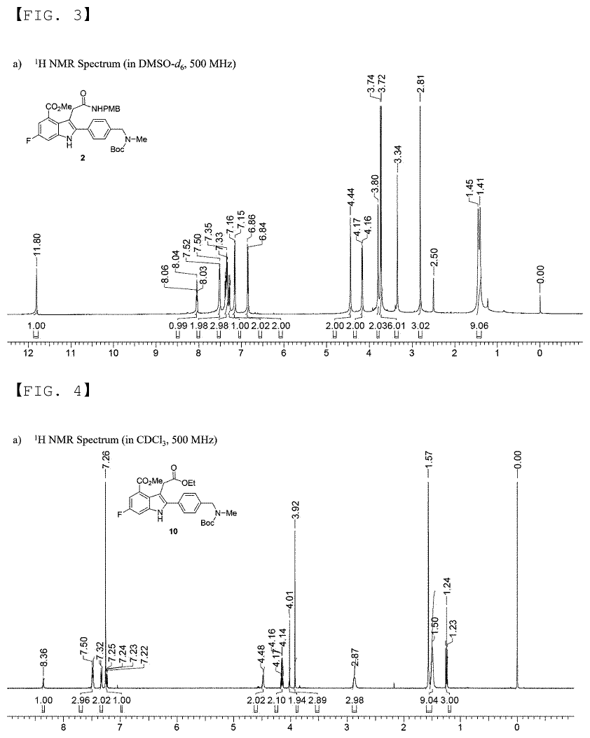

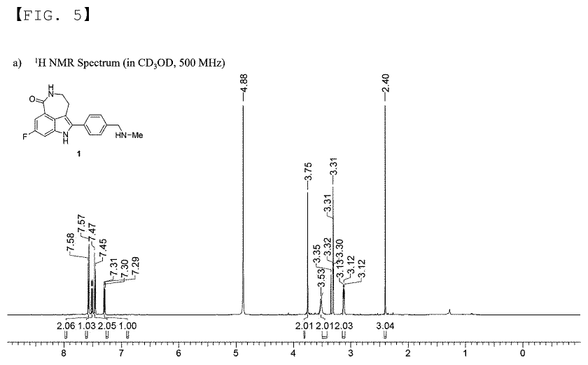

### BEST MODE

Hereinafter, the present disclosure will be described in more detail with reference to examples. However, the following examples are provided to help understand the present disclosure, and the scope of the present disclosure is not limited to the following examples.

### General Procedure

Unless otherwise stated, all reactions were performed in oven-dried glassware under an argon atmosphere. Unless otherwise indicated, all reactions were magnetically stirred, monitored by analytical thin layer chromatography (TLC) using a silica gel glass plate (0.25 mm) pre-coated with an F254 indicator, and visualized with UV light (254 nm). Flash column chromatography was performed using silica gel 60 (230 mesh to 400 mesh) with an indicated eluent. Commercial grade reagents were used without further purification. Unless otherwise stated, a yield refers to chromatographically and spectroscopically pure compound. ¹H NMR and ¹³C NMR spectra were recorded on 500 MHZ and 125 MHZ spectrometers, respectively. Tetramethylsilane (δ_{TMS}: 0.0 ppm) and residual NMR solvent (CDCl₃ (δ_{H}: 7.26 ppm, δ_{C}: 77.16 ppm) or (CD₃)₂SO (δ_{H}: 2.50 ppm, δ_{C}: 39.52 ppm) were used as internal standards for ¹H NMR and ¹³C NMR spectra, respectively. A proton spectrum was expressed as δ(proton position, multiplicity, coupling constant J, number of protons). Multiplicity was expressed as s (singlet), d (doublet), t (triplet), q (quartet), p (quintet), m (multiplet) and br (broad). A high-resolution mass spectrum (HRMS) was recorded on a quadrupole time-of-flight mass spectrometer (QTOF-MS) using electrospray ionization (ESI) as an ionization method.

### Synthesis Example 1. Preparation of Intermediate Compound Using Wittig Reaction

### Synthesis Example 1-1: (4-fluoro-2-methoxycarbonyl-6-nitrobenzyl)triphenylphosphonium bromide (Compound 6)

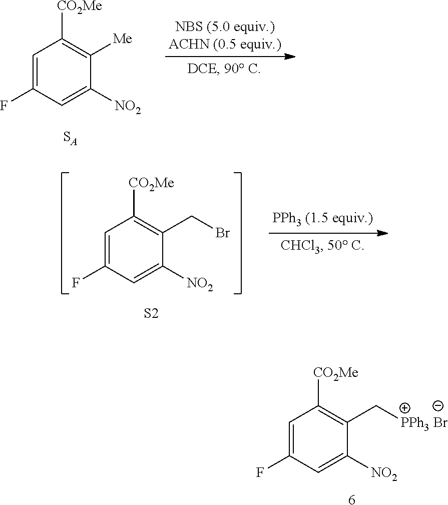

Methyl 5-fluoro-2-methyl-3-nitrobenzoate (S_{A}, 11 g, 50 mmol), N-bromosuccinimide (NBS; 45 g, 250 mmol) and 1,1′-azobis(cyclohexanecarbonitrile) (ACHN; 6.1 g, 25 mmol) were dissolved in 1,2-dichloroethane (DCE; 500 mL), and while stirring the reaction mixture at 90° C., the progress of the reaction was observed by TLC. After Compound S_{A} was completely consumed, the reaction mixture was cooled to 20° C., and a saturated aqueous Na₂S₂O₃ solution (500 ml) was added dropwise to the reaction mixture to remove the remaining N-bromosuccinimide (NBS). After that, the reaction mixture was extracted three times using dichloromethane (500 mL). The obtained organic layer was dried with MgSO₄, and t concentrated to obtain a mixture of Compound S2, and it was directly used in the next reaction without further separation.

After dissolving the mixture of Compound S2 in chloroform (500 mL), triphenylphosphine (20 g, 75 mmol) was added to this mixture solution, and while stirring the reaction mixture at 50° C., the progress of the reaction was observed by TLC. After Compound S2 was completely consumed, the reaction mixture was concentrated, and separated and purified by silica-based column chromatography, in which a mixture solution of dichloromethane and methanol (10:0 to 9:1) was employed as a developing solution, to obtain Compound 5 (25 g, 45 mmol, two-step yield 90%), a dark yellow solid.

¹H NMR (500 MHZ, CDCl₃) δ 7.79-7.72 (m, 10H), 7.68 (dd, J=6.9, 2.7 Hz, 1H), 7.64-7.58 (m, 6H), 5.77 (br, 2H), 3.77 (s, 3H); ¹³C{¹H} NMR (125 MHZ, CDCl₃) δ 164.7, 161.2 (d, J=253.4 Hz), 151.5 (d, J=7.3 Hz), 135.0 (d, J=2.7 Hz), 134.1 (d, J=10.0 Hz), 130.1 (d, J=12.7 Hz), 123.0 (d, J=25.4 Hz), 122.2, 118.7 (d, J=88.2 Hz), 116.5 (d, J=26.3 Hz), 54.1, 26.7 (d, J=52.7 Hz); ¹⁹F NMR (471 MHZ, CDCl₃) δ −106.1; ³¹P NMR (202 MHZ, CDCl₃) δ 25.0; HRMS (ESI-TOF) m/z: [M]⁺ calcd for C₂₇H₂₂FNO₄P 474.1265; found 474.1268.

### Synthesis Example 1-2: (E)-N-(4-Methoxybenzyl)-4-Fluoro-2-Methoxycarbonyl-6-Nitrocinnamide (Compound 8)

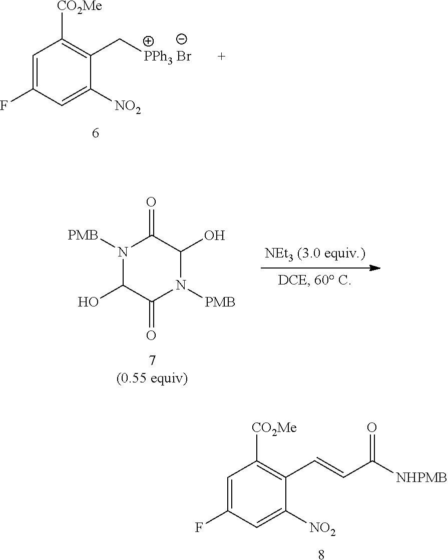

Triethylamine (21 mL, 150 mmol) was added to a 1,2-dichloroethane (500 mL) solution of Compound 6 (28 g, 50 mmol) and Compound 7 (11 g, 27.5 mmol), and while stirring the reaction mixture at 60° C., the progress of the reaction was observed by TLC. After Compound 6 was completely consumed, distilled water (500 mL) was added dropwise to the reaction mixture, and the obtained mixture was extracted three times using dichloromethane (500 mL). The obtained organic layer was dried with MgSO₄ and then concentrated, and separated and purified by silica-based column chromatography, in which a mixture solution of ethyl acetate and hexane (1:2) was employed as a developing solution, to obtain Compound 8 (16.5 g, 42.5 mmol, 85%) as a white solid.

¹H NMR (500 MHZ, CDCl₃) δ 7.95 (d, J=15.9 Hz, 1H), 7.80 (dd, J=8.0, 2.7 Hz, 1H), 7.70 (dd, J=7.2, 2.7 Hz, 1H), 7.24 (d, J=8.5 Hz, 2H), 6.88 (d, J=8.5 Hz, 2H), 5.81 (d, J=15.9 Hz, 1H), 5.76 (br, 1H), 4.47 (d, J=5.5 Hz, 2H), 3.90 (s, 3H), 3.80 (s, 3H); ¹³C{¹H} NMR (125 MHZ, CDCl₃) δ 165.1, 163.9, 161.0 (d, J=254.3 Hz), 159.3, 150.5, 135.3, 134.7 (d, J=7.3 Hz), 129.9, 129.6, 128.1 (d, J=4.5 Hz), 126.5, 121.3 (d, J=22.7 Hz), 114.9 (d, J=26.3 Hz), 114.3, 55.5, 53.4, 43.6; 19F NMR (471 MHZ, CDCl₃) δ −108.2; HRMS (ESI-TOF) m/z: [M+Na]⁺ calcd for C₁₉H₁₇FN₂NaO₆ 411.0963; found 411.0962.

### Synthesis Example 1-3. (E)-N-(4-methoxybenzyl)-2-amino-4-fluoro-6-methoxycarbonylcinnamide (Compound 3)

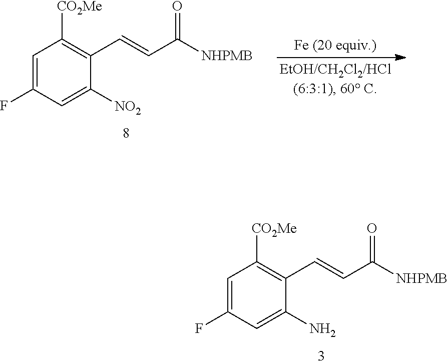

After dissolving Compound 8 (4.3 g, 11 mmol) in a mixture solution of ethanol, dichloromethane and a 35% aqueous hydrochloric acid solution (6:3:1, 180 mL), iron powder (12 g, 220 mmol) was added thereto, and while stirring the reaction mixture at 60° C., the progress of the reaction was observed by TLC. After Compound 8 was completely consumed, the reaction mixture was filtered through Celite to remove insoluble solids, and then concentrated. To the mixture, a saturated aqueous NH₄Cl solution (200 ml) was added dropwise, and then the obtained mixture was extracted three times using ethyl acetate (200 mL). The obtained organic layer was dried with MgSO₄, and then concentrated. The reaction mixture was dissolved again in ethyl acetate, and then hexane was added dropwise thereto to produce white precipitates. The obtained precipitates were filtered to obtain Compound 3 (3.6 g, 10 mmol, 92%), a white solid.

¹H NMR (500 MHZ, DMSO-d₆) δ 8.50 (t, J=5.9 Hz, 1H), 7.49 (d, J=16.0 Hz, 1H), 7.22 (d, J=8.7 Hz, 2H), 6.89 (d, J=8.7 Hz, 2H), 6.65 (dd, J=11.2, 2.7 Hz, 1H), 6.62 (dd, J=8.9, 2.6 Hz, 1H), 6.20 (d, J=16.0 Hz, 1H), 5.73 (s, 2H), 4.30 (d, J=6.0 Hz, 2H), 3.75 (s, 3H), 3.73 (s, 3H); ¹³C{¹H} NMR (125 MHZ, DMSO-d₆) δ 167.6 (d, J=3.6 Hz), 164.7, 161.9 (d, J=242.5 Hz), 158.3, 149.4 (d, J=11.8 Hz), 133.9, 133.7 (d, J=10 Hz), 131.3, 128.8, 125.8, 115.3 (d, J=2.7 Hz), 113.7, 103.4 (d, J=24.5 Hz), 103.3 (d, J=24.5 Hz), 55.1, 52.3, 41.8; 19F NMR (471 MHZ, DMSO-d₆) δ −113.1; HRMS (ESI-TOF): m/z: [M+Na]⁺ calcd for C₁₉H₁₉FN₂NaO₄ 381.1221; found 381.1226.

### Synthesis Example 1-4. Preparation of (E)-ethyl 2-amino-4-fluoro-6-methoxycarbonylcinnamate (Compound 9)

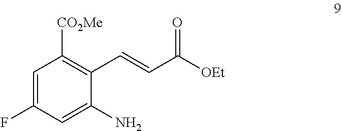 [SMILES: CCOC(=O)/C=C/c1c(N)cc(F)cc1C(=O)OC]

Compound 9 (two-step yield 75%), a yellow solid, was obtained through the process of Synthesis Examples 1-2 and 1-3 using ethyl glyoxylate instead of Compound 7.

¹H NMR (500 MHZ, CDCl₃) δ 7.95 (d, J=16.5 Hz, 1H), 7.00 (dd, J=8.9, 2.6 Hz, 1H), 6.57 (dd, J=9.9, 2.6 Hz, 1H), 6.18 (d, J=16.5 Hz, 1H), 4.27 (q, J=7.2 Hz, 2H), 4.18 (br, 2H), 3.86 (s, 3H), 1.33 (t, J=7.1 Hz, 3H); ¹³C{¹H} NMR (125 MHZ, CDCl₃) δ 166.8 (d, J=3.6 Hz), 166.6, 162.7 (d, J=247.0 Hz), 147.0 (d, J=10.9 Hz), 141.5, 132.8 (d, J=9.1 Hz), 122.7, 116.9 (d, J=2.7 Hz), 107.3 (d, J=24.5 Hz), 105.8 (d, J=24.5 Hz), 60.7, 52.5, 14.3.

### Synthesis Example 1-5. (E)-N-(4-methoxybenzyl)-2-amino-4-fluoro-6-methoxycarbonylcinnamide (Compound 3)

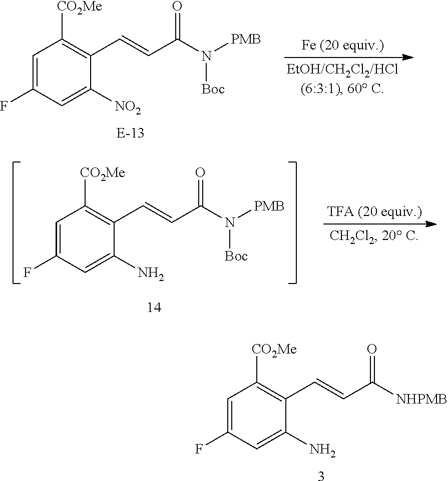

After dissolving Compound E-13 (5.4 g, 11 mmol) in a mixture solution of ethanol, dichloromethane and a 35% aqueous hydrochloric acid solution (6:3:1, 180 mL), iron powder (12 g, 220 mmol) was added thereto, and while stirring the reaction mixture at room temperature, the progress of the reaction was observed by TLC. After Compound E-13 was completely consumed, the reaction mixture was filtered through Celite to remove insoluble solids, and then concentrated. To the mixture, a saturated aqueous NH₄Cl solution (200 ml) was added dropwise, and then the obtained mixture was extracted three times using ethyl acetate (200 mL). The obtained organic layer was dried with MgSO₄, and then concentrated to obtain a mixture of Compound 14 and Compound 3 (8/9=5:1), and it was directly used in the next reaction without further separation.

After dissolving the mixture of Compound 14 and Compound 3 in dichloromethane (110 mL), trifluoroacetic acid (TFA; 17 mL, 220 mmol) was added to this mixture solution, and while stirring the reaction mixture at room temperature, the progress of the reaction was observed by TLC. After Compound 14 was completely converted into Compound 3, distilled water (110 mL) was added dropwise to the reaction mixture, and the obtained mixture was extracted three times using dichloromethane (110 mL). The obtained organic layer was dried with MgSO₄, and then concentrated. The reaction mixture was dissolved again in ethyl acetate, and then hexane was added dropwise thereto to produce white precipitates. The obtained precipitates were filtered to obtain Compound 3 (3.6 g, 10 mmol, two-step yield 92%), a white solid.

¹H NMR (500 MHZ, DMSO-d₆) δ 8.50 (t, J=5.9 Hz, 1H), 7.49 (d, J=16.0 Hz, 1H), 7.22 (d, J=8.7 Hz, 2H), 6.89 (d, J=8.7 Hz, 2H), 6.65 (dd, J=11.2, 2.7 Hz, 1H), 6.62 (dd, J=8.9, 2.6 Hz, 1H), 6.20 (d, J=16.0 Hz, 1H), 5.73 (s, 2H), 4.30 (d, J=6.0 Hz, 2H), 3.75 (s, 3H), 3.73 (s, 3H); ¹³C{¹H} NMR (125 MHZ, DMSO-d₆) δ 167.6 (d, J=3.6 Hz), 164.7, 161.9 (d, J=242.5 Hz), 158.3, 149.4 (d, J=11.8 Hz), 133.9, 133.7 (d, J=10 Hz), 131.3, 128.8, 125.8, 115.3 (d, J=2.7 Hz), 113.7, 103.4 (d, J=24.5 Hz), 103.3 (d, J=24.5 Hz), 55.1, 52.3, 41.8; 19F NMR (471 MHZ, DMSO-d₆) δ −113.1; HRMS (ESI-TOF): m/z: [M+Na]⁺ calcd for C₁₉H₁₉FN₂NaO₄ 381.1221; found 381.1226.

### Synthesis Example 2: N-(4-methoxybenzyl)-2-(4-(N-tert-butoxycarbonyl-N-methylaminomethyl)phenyl)-6-fluoro-4-methoxycarbonyl-indole-3-acetamide (Compound 2)

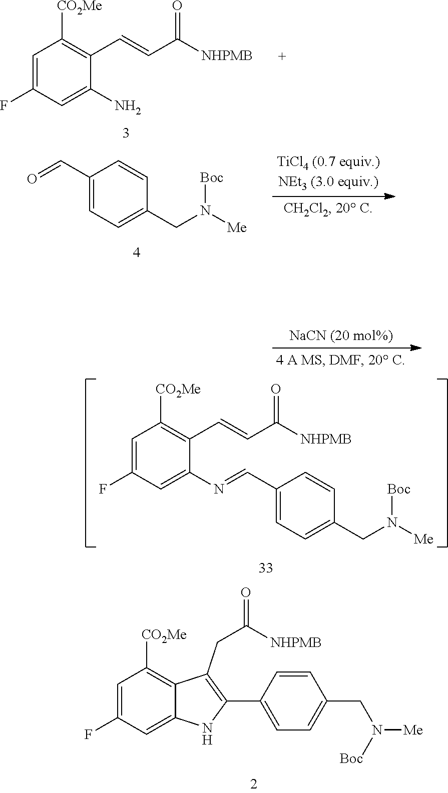

Titanium tetrachloride (1.0 M dichloromethane solution, 7.0 mL, 7.0 mmol) was added to a dichloromethane (100 mL) solution of 2-aminocinnamide Compound 3 (3.6 g, 10 mmol), aldehyde Compound 4 (2.5 g, 10 mmol) and triethylamine (4.2 mL, 30 mmol), and while stirring the reaction mixture at 20° C., the progress of the reaction was observed by TLC and ¹H NMR analysis. After Compound 3 and Compound 4 were completely consumed, distilled water (100 mL) was added dropwise to the reaction mixture, and the obtained mixture was extracted three times using dichloromethane (100 mL). The obtained organic layer was dried with MgSO₄, and then concentrated to obtain a mixture of Compound S3, and it was directly used in the next reaction without further separation.

After dissolving the mixture of Compound S3 in dimethylformamide (100 mL), 4 Å molecular sieves (3.0 g) and sodium cyanide (98 mg, 2.0 mmol) were added to this mixture solution, and while stirring the reaction mixture at 20° C., the progress of the reaction was observed by TLC. After Compound S3 was completely consumed, insoluble solids were filtered and removed, and the filtrate was washed with ethyl acetate. The obtained filtrate was concentrated, and then the reaction mixture was separated and purified by silica-based column chromatography, in which a mixture solution of ethyl acetate and hexane (1:2) was employed as a developing solution, to obtain Compound 2 (4.7 g, 8.0 mmol, two-step yield 80%), a yellow solid.

¹H NMR (500 MHZ, DMSO-d₆) δ 11.80 (s, 1H), 8.04 (t, J=5.9 Hz, 1H), 7.51 (d, J=7.5 Hz, 2H), 7.37 (m, 1H), 7.34 (d, J=7.8 Hz, 2H), 7.28 (m, 1H), 7.16 (d, J=8.2 Hz, 2H), 6.85 (d, J=8.2 Hz, 2H), 4.44 (s, 2H), 4.17 (d, J=5.8 Hz, 2H), 3.80 (s, 2H), 3.74 (s, 3H), 3.72 (s, 3H), 2.81 (s, 3H), 1.46-1.40 (br, 9H); ¹³C{¹H} NMR (125 MHZ, DMSO-d₆) δ 171.0, 167.0 (d, J=2.7 Hz), 158.1, 157.1 (d, J=235.2 Hz), 139.5, 138.3, 137.3 (d, J=11.8 Hz), 131.9, 130.8, 128.9, 128.5, 127.5, 124.7 (d, J=8.2 Hz), 123.1, 113.5, 109.6 (d, J=25.4 Hz), 106.0, 101.1 (d, J=24.5 Hz), 78.9, 55.0, 52.1, 50.8, 41.7, 34.0, 32.9, 28.1; ¹⁹F NMR (471 MHZ, DMSO-d₆) δ −122.4; HRMS (ESI-TOF): m/z [M+Na]⁺ calcd for C₃₃H₃₆FN₃NaO₆ 612.2480; found 612.2482.

### Synthesis Example 3-1: Ethyl 2-(4-(N-tert-butoxycarbonyl-N-methylaminomethyl)phenyl)-6-fluoro-4-methoxycarbonyl-indole-3-acetate (Compound 10)

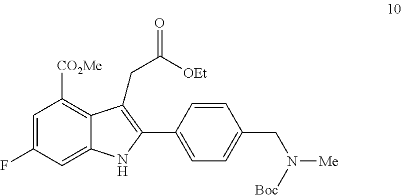 [SMILES: CCOC(=O)Cc1c(-c2ccc(CN(C)C(=O)OC(C)(C)C)cc2)[nH]c2cc(F)cc(C(=O)OC)c12]

Compound 10 (two-step yield 88%), a yellow solid, was obtained through the process of Synthesis Example 2 using Compound 9 instead of Compound 3.

¹H NMR (500 MHZ, CDCl₃) δ 8.36 (br, 1H), 7.52-7.45 (m, 3H), 7.33 (d, J=7.9 Hz, 2H), 7.24 (dd, J=8.5, 2.4 Hz, 1H), 4.48 (br, 2H), 4.15 (q, J=7.2 Hz, 2H), 4.01 (s, 2H), 3.92 (s, 3H), 2.87 (br, 3H), 1.50 (br, 9H), 1.24 (t, J=7.1 Hz, 3H); ¹³C{¹H} NMR (125 MHZ, CDCl₃) δ 173.4, 167.7, 158.2 (d, J=238.9 Hz), 156.2 (d, J=60.8 Hz), 139.6, 138.5 (d, J=30.9 Hz), 137.4 (d, J=11.8 Hz), 131.0, 129.1, 127.8, 124.6, 123.4, 111.7 (d, J=24.5 Hz), 105.9, 101.8 (d, J=25.4 Hz), 80.2, 60.7, 52.3, 52.2 (d, J=76.3 Hz), 34.4 (d, J=33.6 Hz), 32.8, 28.6, 14.3.

### Synthesis Example 3-2: N-(4-methoxybenzyl)-2-(4-(N-tert-butoxycarbonyl-N-methylaminomethyl)phenyl)-6-fluoro-4-methoxycarbonyl-indole-3-acetamide (Compound 2)

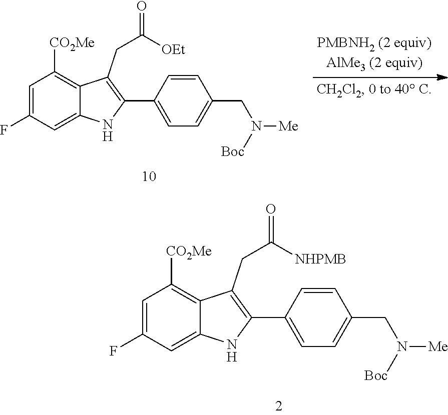

After adding trimethylaluminum (2.0 M hexane solution, 1.0 mL, 2.0 mmol) to a dichloromethane (10 mL) solution of 4-methoxybenzylamine (0.26 mL, 2.0 mmol) at 0° C., the reaction mixture was stirred for 30 minutes. Compound 10 (0.49 g, 1.0 mmol) was added to this reaction mixture, and while stirring the reaction mixture at 40° C., the progress of the reaction was observed by TLC. After Compound 10 was completely consumed, the reaction mixture was cooled to 0° C., and a 1.0 N aqueous hydrochloric solution (10 mL) was added dropwise thereto to remove the remaining trimethylaluminum. After that, the reaction mixture was extracted three times using dichloromethane (10 mL). The obtained organic layer was dried with MgSO₄, and then concentrated. The obtained reaction mixture was separated and purified by silica-based column chromatography, in which a mixture solution of ethyl acetate and hexane (1:3) was employed as a developing solution, to obtain Compound 2 (0.29 g, 0.50 mmol, 50%), a yellow solid.

### Synthesis Example 4: Preparation of Rucaparib (Compound 1)

### Synthesis Example 4-1: N-(4-methoxybenzyl)-N-(tert-butoxycarbonyl) rucaparib (Compound 12)

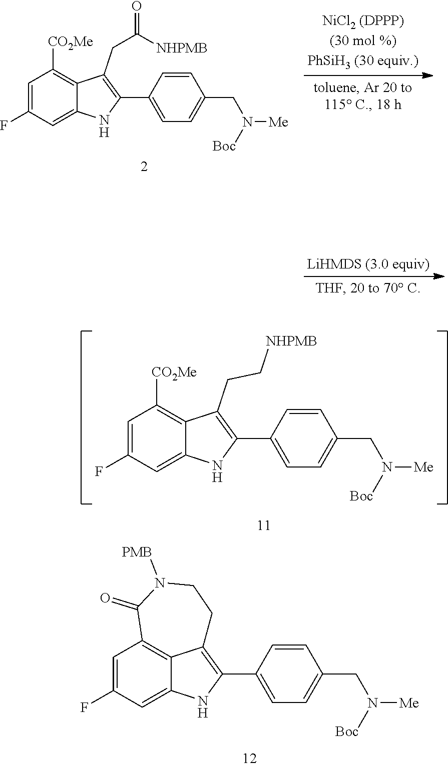

Phenylsilane (1.2 mL, 10 mmol) was added to a toluene (10 mL) solution of Compound 2 (590 mg, 1.0 mmol) and NiCl₂ (DPPP) (DPPP=1,3-bis(diphenylphosphino) propane) (162 mg, 0.30 mmol) at 20° C., and then the reaction mixture was stirred at 115° C. After that, additional phenylsilane (1.2 mL, 10 mmol) was added dropwise to the reaction mixture twice at an interval of 2 hours, and the reaction mixture was further stirred for 14 hours at 115° C. After Compound 2 was completely consumed, the reaction mixture was cooled to 0° C., and a 1.0 N aqueous NaOH solution (20 mL) was added dropwise thereto, Then, the obtained mixture was extracted three times using ethyl acetate (20 mL). The obtained organic layer was dried with MgSO₄, and then concentrated to obtain a mixture of Compound 11, and it was directly used in the next reaction without further separation.

After dissolving the mixture of Compound 11 in tetrahydrofuran (10 mL), lithium bis(trimethylsilyl)amide (1.0 M tetrahydrofuran solution, 3.0 mL, 3.0 mmol) was added to this mixture solution at 20° C., and while stirring the reaction mixture at 70° C., the progress of the reaction was observed by TLC. After Compound 11 was completely consumed, distilled water (10 mL) was added dropwise to the reaction mixture, and the obtained mixture was extracted three times using ethyl acetate (10 mL). The obtained organic layer was dried with MgSO4 and then concentrated, and separated and purified by silica-based column chromatography, in which a mixture solution of ethyl acetate and hexane (1:2) was employed as a developing solution, to obtain Compound 12 (451 mg, 0.83 mmol, 83%) as a yellow solid.

Compound 12: ¹H NMR (500 MHZ, CDCl₃) δ 8.92 (s, 1H), 7.79 (dd, J=2.2, 10.9 Hz, 1H), 7.50-7.39 (m, 2H), 7.32 (d, J=8.5 Hz, 2H), 7.28 (br, 2H), 7.19 (d, J=7.9 Hz, 1H), 6.88 (d, J=8.5 Hz, 2H), 4.94-4.72 (m, 2H), 4.44 (s, 2H), 3.81 (s, 3H), 3.62 (br, 2H), 2.95 (br, 2H), 2.83 (br, 3H), 1.49 (d, J=12.5 Hz, 9H); ¹³C{¹H} NMR (125 MHZ, CDCl₃) δ 168.5 (d, J=1.8 Hz), 159.7 (d, J=238.9 Hz), 159.1, 156.2 (d, J=55.4 Hz), 137.8 (d, J=17.3 Hz), 136.6 (d, J=11.8 Hz), 135.1, 130.8, 129.73, 129.71, 128.0, 127.9, 126.5 (d, J=8.2 Hz), 123.7, 114.2, 112.4, 112.2 (d, J=26.3 Hz), 101.1 (d, J=26.3 Hz), 80.1, 55.4, 52.5, 51.8, 49.0, 34.3, 28.6, 28.0; 19F NMR (471 MHZ, CDCl₃) δ −120.1; HRMS (ESI-TOF) m/z: [M+Na]⁺ calcd for C₃₂H₃₄FN₃NaO₄ 566.2426; found: 566.2429.

### Synthesis Example 4-2: Preparation of Rucaparib (Compound 1)

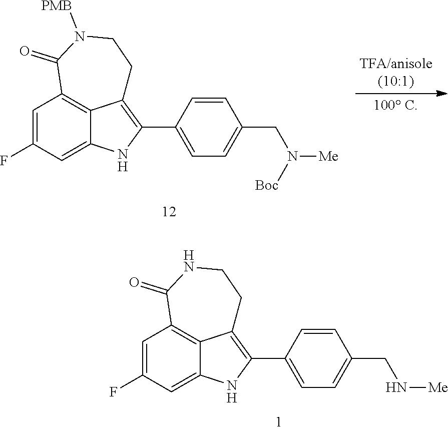

Compound 12 (270 mg, 0.5 mmol) was dissolved in a mixture solution of trifluoroacetic acid and anisole (10:1, 5.0 mL), and while stirring the reaction mixture at 100° C., the progress of the reaction was observed by TLC. After Compound 12 was completely consumed, the reaction mixture was concentrated, and separated and purified by silica-based column chromatography, in which a mixture solution of dichloromethane, methanol and triethylamine (90:10:1) was employed as a developing solution, to obtain rucaparib (Compound 1) (160 mg, 0.49 mmol, 98%), a white solid.

¹H NMR (500 MHZ, CD₃OD) δ 7.57 (d, J=8.2 Hz, 2H), 7.51 (dd, J=10.8, 2.3 Hz, 1H), 7.46 (d, J=8.2 Hz, 2H), 7.30 (dd, J=9.0, 2.4 Hz, 1H), 3.75 (s, 2H), 3.53 (br, 2H), 3.15-3.10 (m, 2H), 2.40 (s, 3H); ¹³C{¹H} NMR (125 MHZ, CD₃OD) δ 172.6, 160.6 (d, J=235.2 Hz), 140.2, 138.6 (d, J=11.8 Hz), 137.3 (d, J=3.6 Hz), 132.3, 130.0, 129.2, 125.8 (d, J=9.1 Hz), 125.0, 112.9, 111.2 (d, J=26.3 Hz), 102.2 (d, J=26.3 Hz), 56.0, 43.8, 35.6, 30.0; 19F NMR (471 MHZ, CD₃OD) δ −123.4; HRMS (ESI-TOF) m/z: [M+H]⁺ calcd for C₁₉H₁₉FN₃O 324.1507; found: 324.1510.

1. A method for preparing a compound of Chemical Formula (3), the method comprising:
reacting a compound of the following Chemical Formula (1) and a compound of the following Chemical Formula (2); and
converting the result into a compound of the following Chemical Formula (3) in the presence of a catalyst: 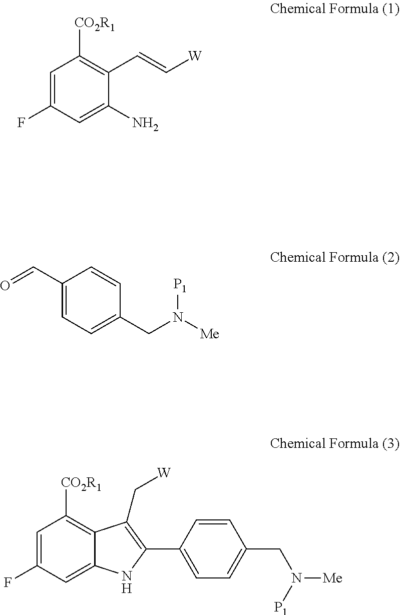
herein,
R₁ is a linear or branched C₁-C₅ alkyl;
W is selected from the group consisting of —COOR₂, —CONH₂, —CONHP₂ and —CONP₂P₃;
R₂ is H or a linear or branched C₁-C₅ alkyl; and
P₁, P₂ and P₃ are, as an amine protecting group, each independently selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

2. The method of claim 1, wherein the conversion into the compound of Chemical Formula (3) is performed in the presence of a dehydrating agent.

3. The method of claim 2, wherein the dehydrating agent is at least one compound selected from the group consisting of TiCl₄, MgSO₄ and Na₂SO₄, or a molecular sieve.

4. The method of claim 1, wherein the conversion into the compound of Chemical Formula (3) is performed using an azeotropic distillation method.

5. The method of claim 1, wherein the catalyst is MCN or N-heterocyclic carbene:
herein,
M is an alkali metal or NR₄⁺; and
R is H or a linear or branched C₁-C₅ alkyl.

6. The method of claim 5, wherein the N-heterocyclic carbene is selected from the group consisting of imidazolium, triazolium and thiazolium.

7. The method of claim 1, further comprising, when W is —COOR₂, —CONH₂ or —CONP₂P₃, converting the —COOR₂, —CONH₂ or —CONP₂P₃ into —CONHP₂ after the conversion to the compound of Chemical Formula (3):
herein, R₂ is H or a linear or branched C₁-C₅alkyl; and
P₁, P₂ and P₃ are, as an amine protecting group, each independently selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

8. A method for preparing rucaparib, a compound of the following Chemical Formula (5), the method comprising:
(a) reacting a compound of the following Chemical Formula (1) and a compound of the following Chemical Formula (2), and converting the result to a compound of the following Chemical Formula (3) in the presence of a catalyst, however, when W is —COOR₂, —CONH₂ or —CONP₂P₃, converting the —COOR₂, —CONH₂ or —CONP₂P₃ into —CONHP₂ is further included after (a);
(b) performing a reaction of reducing the compound of Chemical Formula (3) to convert the result into a compound of the following Chemical Formula (4); and
(c) subjecting the compound of Chemical Formula (4) to a lactam ring formation reaction, and to deprotection before, after or simultaneously with the reaction to obtain compound of the following Chemical Formula (5): 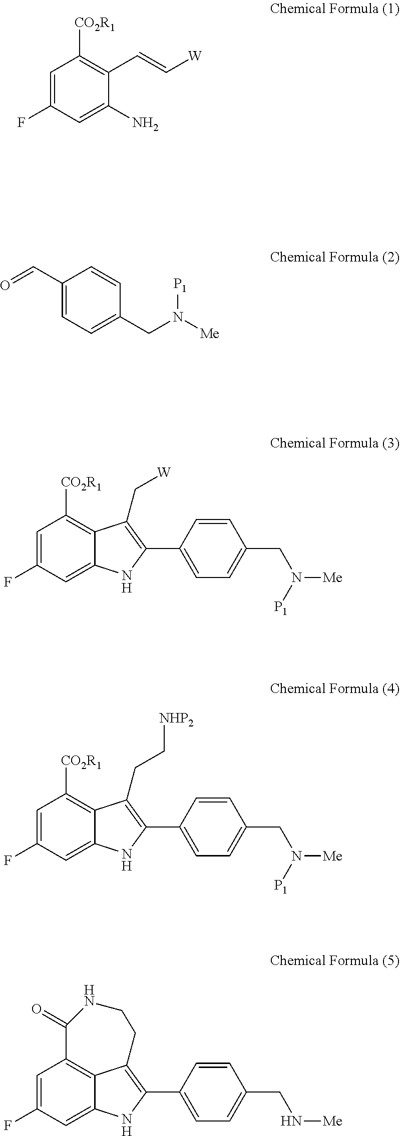
herein,
R₁ is a linear or branched C₁-C₅ alkyl;
W is selected from the group consisting of —COOR₂, —CONH₂, —CONHP₂ and —CONP₂P₃;
R₂ is H or a linear or branched C₁-C₅ alkyl; and
P₁, P₂ and P₃ are, as an amine protecting group, each independently selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

9. The method of claim 8, wherein (a) is performed in the presence of a dehydrating agent.

10. The method of claim 9, wherein the dehydrating agent is at least one compound selected from the group consisting of TiCl₄, MgSO₄ and Na₂SO₄, or a molecular sieve.

11. The method of claim 8, wherein (a) is performed using an azeotropic distillation method.

12. The method of claim 8, wherein the catalyst used in (a) is MCN or N-heterocyclic carbene:
herein, M is an alkali metal or NR₄⁺; and
R is H or a linear or branched C₁-C₅ alkyl.

13. The method of claim 8, wherein the reduction reaction of (b) is performed in the presence of a metal catalyst selected from the group consisting of Ni, Zn, Fe and Co, and a silane compound.

14. The method of claim 8, wherein the reduction reaction of (b) is performed in the presence of a metal hydride selected from the group consisting of DIBAL-H, L-selectride, NaBH₄ and borane.

15. The method of claim 8, wherein the lactam ring formation reaction of (c) is performed in the presence of a base.

16. A method for preparing a compound of Chemical Formula (1), the method comprising:
(a′) converting a compound of the following Chemical Formula (6) to a compound of the following Chemical Formula (7);
(b′) reacting the compound of Chemical Formula (7) with a compound of the following Chemical Formula (8) to prepare a compound of the following Chemical Formula (9); and
(c′) performing a reaction of reducing the compound of Chemical Formula (9): 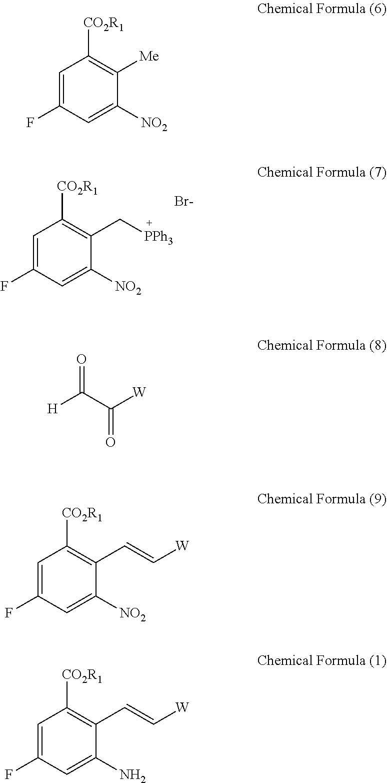
herein,
R₁ is a linear or branched C₁-C₅ alkyl;
W is selected from the group consisting of —COOR₂, —CONH₂, —CONHP₂ and —CONP₂P₃;
R₂ is H or a linear or branched C₁-C₅ alkyl; and
P₁, P₂ and P₃ are, as an amine protecting group, each independently selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

17. A compound of the following Chemical Formula (1) used in preparation of rucaparib: 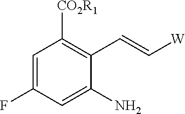
herein,
R₁ is a linear or branched C₁-C₅ alkyl;
W is selected from the group consisting of —COOR₂, —CONH₂, —CONHP₂ and —CONP₂P₃;
R₂ is H or a linear or branched C₁-C₅ alkyl; and
P₁, P₂ and P₃ are, as an amine protecting group, each independently selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).

18. A compound of the following Chemical Formula (3) used in preparation of rucaparib: 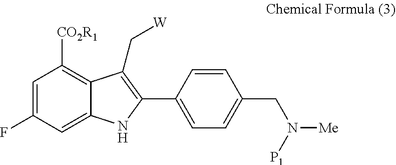
herein,
R₁ is a linear or branched C₁-C₅ alkyl;
W is selected from the group consisting of —COOR₂, —CONH₂, —CONHP₂ and —CONP₂P₃;
R₂ is H or a linear or branched C₁-C₅ alkyl; and
P₁, P₂ and P₃ are, as an amine protecting group, each independently selected from the group consisting of methoxycarbonyl, ethoxycarbonyl, diisopropylmethoxycarbonyl, t-butyloxycarbonyl (Boc), carbobenzyloxy (Cbz), 9-fluorenylmethyloxycarbonyl (Fmoc), acetyl (Ac), benzoyl (Bz), benzyl (Bn), p-methoxybenzyl (PMB), 3,4-dimethoxybenzyl (DMPM), p-methoxyphenyl (PMP), tosyl (Ts), 2,2,2-trichloroethoxycarbonyl (Troc), 2-trimethylsilylethoxycarbonyl (Teoc) and aryloxycarbonyl (Alloc).
